(19) United States
(12) Patent Application Publication
(10) Pub. No.: US 2010/0094266 A1
(43) Pub. Date: Apr. 15, 2010

(54) LASER ACTIVATED MICRO ACCELERATOR PLATFORM

(75) Inventors: Gil Travish, Santa Monica, CA (US); Rodney B. Yoder, White Plains, NY (US); James Rosenzweig, Los Angeles, CA (US)

Correspondence Address: JOHN P. O'BANION;O'BANION & RITCHEY LLP, 400 CAPITOL MALL SUITE 1550, SACRAMENTO, CA, 95814, US

(73) Assignee: THE REGENTS OF THE UNIVERSITY OF CALIFORNIA, Oakland, CA (US)

(21) Appl. No.: 12/570,837

(22) Filed: Sep. 30, 2009

Related U.S. Application Data
(63) Continuation of application No. PCT/US08/59478, filed on Apr. 4, 2008.
(60) Provisional application No. 60/910,090, filed on Apr. 4, 2007.

(51) Int. Cl. A61B 18/24; H01J 35/32; H05H 7/00

(52) U.S. Cl. 606/15; 378/121; 315/507

(57) ABSTRACT

A resonant laser powered micro accelerator platform capable of producing relativistic or near relativistic electrons and, optionally, x-rays. The apparatus has a pair of parallel slab-symmetric dielectric slabs that are separated by a narrow vacuum gap that is preferably tapered. The slabs have a top surface with reflective layers with many periodic slots creating longitudinal periodicity in the structure fields when laser light is directed on the reflectors in one embodiment. Electrons introduced into the gap are accelerated along the length of the slabs. The reflective surface of the slabs is preferably a laminate of alternating layers of high index and low index of refraction materials.

### CROSS-REFERENCE TO RELATED APPLICATIONS

This application claims priority from, and is a 35 U.S.C. §111(a) continuation of, PCT international application serial number PCT/US2008/059478, filed on Apr. 4, 2008, incorporated herein by reference in its entirety, which claims priority from U.S. provisional application Ser. No. 60/910,090 filed on Apr. 4, 2007, incorporated herein by reference in its entirety.

This application is also related to PCT International Publication No. WO 2008/156896 published on Dec. 24, 2008 and republished on Feb. 19, 2009, incorporated herein by reference in its entirety.

### STATEMENT REGARDING FEDERALLY SPONSORED RESEARCH OR DEVELOPMENT

This invention was made with Government support under Grant No. DE-FG02-92ER40693, awarded by the Department of Energy. The Government has certain rights in this invention.

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to ionizing radiation sources, and more particularly to a self contained micro scale laser activated electron accelerator platform for producing relativistic and near relativistic electrons or bremsstrahlung x-rays. Ionizing radiation produced by the device and its small size make the apparatus of the invention particularly useful for minimally invasive laser endoscopic medical procedures.

2. Description of Related Art

The application of ionizing radiation is a proven method of treatment of tumors and other cellular proliferative disorders in mammals. Although certain types of cancer can be treated successfully with current methods, cancer remains a major disease and continues to be a leading cause of death.

Most cancer patients that are treated with radiation therapy are exposed to an external high energy radiation beam, typically electrons or x-rays, from a large external linear accelerator. The precision attained using such methods can be very high, especially with the use of PET- or CAT-aided sources with computer control to produce precisely-directed radiation beams (“stereotactic radiosurgery”). While most of these radiation treatments are of cancers, radiosurgery is also a treatment option for other uncommon conditions such as arteriovenous malformation (AVS) in the brain. The energy of the radiation used for these medical purposes varies according to the method used but typically range from 6 MeV to 12 MeV.

However, external ionizing radiation sources deliver undesirable radiation doses to surrounding healthy tissues because the beams must be directed through large sections of healthy organs and tissues to reach the target tissues. Secondary damage skin, bones, internal organs and other healthy tissues is a significant undesirable side effect from radiation therapy. Therefore, the challenge of external radiation therapy is to maximize the delivery of a therapeutic radiation dose to the target tumor tissue while minimizing the radiation exposure to healthy surrounding tissues.

In a related procedure known as intra-operative radiation therapy (IORT), a short burst of radiation is delivered to the tumor site during surgery itself. The radiation can be from an external x-ray or electron beam (produced by a large linear accelerator) as well as small radioactive sources. Typical IORT cases involve tumors that cannot be completely removed with safety, and include breast cancer (lumpectomy surgery), rectal/colon cancer, recurrent forms of gynecological and urinary cancers, head and neck tumors, and soft-tissue sarcoma.

The placement of internal radiation sources for radiation therapy has been developed to reduce the negative effects of external radiation therapy on intervening healthy tissues. For example, patients may be treated with internal exposures to radioactive substances that are implanted or delivered through a catheter. Brachytherapy, also known as internal radiation therapy or IRT, is a specialized option in the treatment for certain cancers. Typically, radioactive source material is introduced directly into the body, in the form of “seeds” or pellets of radioactive material (e.g. iridium-192 or strontium-90). In some cases of prostate cancer treatment, low-energy radiation is produced over a period of weeks or months from hundreds of seeds inserted into a tumor. Most conventional brachytherapy produces low-energy photons or beta particles, on the order of 50 keV. The penetration of these particles into the surrounding tissue is therefore very limited, which may be of benefit to the healthy tissues of the patient.

Another type of brachytherapy is high dose-rate (HDR) brachytherapy, in which a much higher dose is obtained for a short time (tens of minutes, repeated over several days). This is often accomplished by introducing radioactive isotopes by a catheter into the affected region, and withdrawing them again when the dose is complete. Another version of HDR brachytherapy is illustrated in one post-operative treatment of breast cancer. In one system, a liquid filled balloon catheter is inflated in the space left by the tumor's removal, and radioactive isotopes are used to deliver a high but localized radiation dose to the tissue surrounding the tumor location. The cancers treated by these methods are usually near the body surface or near an orifice.

Brachytherapy has also been used following angioplasty for the treatment of cardiovascular disease. When plaque is removed from a coronary artery via percutaneous coronary intervention (PCI), a tube-like wire mesh stent is usually inserted into the artery to maintain its shape. To prevent in-stent restenosis, in which the artery recloses due to the growth of abnormal cells within the stent, the site may be treated with radiation (“vascular brachytherapy”), typically produced by radioisotopes that are introduced via catheter into the artery.

The use of radioactive isotopes as a source of ionizing radiation is accompanied with a number of risks and disadvantages. First, implanted radioactive sources will continue to emit ionizing radiation, often beyond the life of the patient, and create a risk of damage to healthy tissue over a time. Physicians and other hospital staff that handle the radioactive materials may be exposed to ionizing radiation over time. There is also the further administrative burden associated with obtaining, maintaining, and disposing of radioactive materials. Therefore, implanted radioactive materials are undesirable because they cannot be turned off and on, are complicated to use, and must be well shielded and controlled for safe operation.

Several devices have been developed to avoid the placement of radioactive materials in a patient and to reduce the deposition of radiation in healthy organs and tissues from external radiation sources. For example, one commercial device uses a miniaturized x-ray tube to deliver 50-kV x-ray bursts within the body. Miniature x-ray tubes avoid the issues surrounding radioactive materials, but are limited to a very specific energy range (10-50 kV), and do not have the ability to select or collimate the beam that is produced. The produced spectrum is broad and peaked at low energies, and the beam is likewise spread over a wide angle.

The use of x-ray technology may also require the introduction of high voltage (50 kV) directly into the body of the patient in order to power the x-ray tube. Although these devices are miniaturized, the x-ray tubes still measure several millimeters in each direction and (because of the need for voltage isolation) must be mounted in a rigid and thick support rather than on a narrow catheter limiting its usefulness.

Another problem observed with miniature x-ray tube generators is the generation of excessive heat by the anode of the tube. Excessive heat may also damage surrounding healthy tissues or blood vessels. Other miniature x-ray tube designs have the tube within an inflatable balloon that can provide some thermal insulation and circulating fluids to eliminate heat. However, these designs still require the creation of large voltages within the body to activate the device and are bulky.

Further internal designs provide a flexible x-ray radiation transmitting needle with x-rays or electrons transmitted through hollow glass fibers or other reflective beam transmitting tubes. The needle tip is introduced into the tumor or other tissue and radiation is delivered to the site through the needle. However, there is a large loss in radiation intensity due to the reflections making longer exposure times with minimal therapeutic radiation exposure.

Accordingly, there is a need for the development of micro-sized devices that can treat interior cancers with ionizing radiation from a source located at or near the target tumor site that minimizes the exposure of adjacent organs and tissues to radiation. There is also a need for a catheter positioned device that provides ionizing radiation to a target tissue site that does not require the introduction of large voltages, excessive heat or radioactive materials into the body. There is a further need for a micro-device that will provide controlled exposures of target tissues to ionizing radiation of selected intensities and durations that does not expose the medical staff or the patient to hazardous materials or require radiation safety protocols. There is also a need for a device that is comparatively inexpensive to manufacture, easy to use, and adaptable to a wide variety of therapeutic oncology treatments in inaccessible organs, coronary stent implantation, the destruction of AVM abnormalities and other uses. The present invention satisfies these needs as well as others and is a general improvement over the devices and treatments in the art.

### BRIEF SUMMARY OF THE INVENTION

The present invention is a micro-scale resonant laser powered structure that can generate and accelerate electrons or generate x-rays. One adaptation of the invention is a medical device which is able to deliver therapeutic doses of ionizing radiation directly to organs, tumors, or blood vessels within the body. The radiation produced consists of pulses of relativistic electrons (beta particles) of energy about 1 MeV to about 5 MeV. This radiation production is accomplished by a sub-millimeter-sized electron accelerator, which can be mounted in a fiber-optic catheter and can be inserted laparoscopically into tissues or organs. This device is particularly suited for performing medical brachytherapy, in which therapeutic radiation is delivered directly to the desired location by a small and localized radiation source introduced into the body. However, the apparatus may be used in any setting where accelerated electrons or x-rays are needed.

Brachytherapy is not limited to any single medical purpose or procedure. Several different forms of this therapy can be used for treatment of superficial tumors and cancers of accessible organs such as the prostate, cervix, breast, head and neck, and lungs. In a related application, a tumor bed can be irradiated immediately following surgical removal of the tumor (interoperative radiation therapy, or IORT). Brachytherapy has also been used during the installation of arterial stents during the treatment of coronary artery disease, where it can prevent the re-closing of the blood vessel around the stent without the use of drugs.

The invention contains no radioactive isotopes; the radiation produced is in a narrow beam that is turned on only during brief pulses. There is no radiation anywhere in the device and no need for shielding when it is not active. The electron beam produced has a relatively narrow energy peak which can be selected during manufacture.

According to one aspect of the invention, a radiation source is provided that has an evacuated housing containing a micro-accelerator platform assembly with a pair of dielectric slabs separated by a vacuum gap, each slab having at reflective layer on a side opposite said gap, with at least one reflective layer having a plurality of periodic slots and an active surface. An optical source adapted to directing beams of light to the reflective layers of the dielectric slabs and a source of electrons configured to emit electrons within said vacuum gap and accelerated.

According to another aspect of the invention, a micro-accelerator platform is provided that has an electron source, a first dielectric slab with a reflective surface having a plurality of slots and an active surface and a second dielectric slab with a reflective surface having a plurality of slots and an active surface that is oriented opposite said active surface of the first dielectric slab forming a gap between the active surfaces. A source of optical radiation is configured to direct beams of light on the reflective surfaces of the first and second dielectric slabs and electrons emitted from the electron source are accelerated within said gap between the active surfaces of the two dielectric slabs.

Another aspect of the invention is to provide a micro-accelerator platform that has a first dielectric slab with a reflective surface having a plurality of slots and an active surface and a second dielectric slab with a reflective surface and an active surface with the active surfaces oriented opposite each other forming a gap between the active surfaces. The reflective surface of the second slab can be a metal reflector. A source of optical radiation directs beams of light on the slotted reflective surface of the first dielectric slab and an electron source emits electrons within the gap that are then accelerated.

In a further aspect of the invention, a radiation source is provided that has an electron source that has a ferroelectric crystal base, an emitter array coupled to the ferroelectric crystal base; and a heating element. The emitter array preferably is made of graphite needles and the ferroelectric crystal base is preferably made from lithium niobate.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a side schematic view of one micro-accelerator platform embodiment with symmetrically paired dielectric wafers/slabs according to the invention.

FIG. 2 is a side schematic view of an alternative micro-accelerator platform embodiment with paired dielectric wafers/slabs with one reflecting slab surface containing periodic coupling slots and the other dielectric slab disposed on a simple reflecting surface according to the invention.

FIG. 3 is a schematic view of the paired periodic slab structure of the embodiment shown in FIG. 1.

FIG. 4A is a schematic side view of one embodiment of the slab structure detailing the alternating layers of high and low refractive index materials and slots.

FIG. 4B is a schematic top view of one embodiment of the slab structure detailing the periodic slots according to the present invention.

FIG. 5 is a schematic top view of an alternative embodiment of a slab structure with the coupling slots rotated slightly from perpendicular and alternating in sign every few structure periods.

FIG. 6 is a schematic view of one embodiment of an integrated particle emitter according to the invention.

FIG. 7A is a graph of particle energy along the structure of a simulated accelerator.

FIG. 7B is a graph of focusing a canted-slot configuration showing the values of x and y in the first 20 periods of the structure.

### DETAILED DESCRIPTION OF THE INVENTION

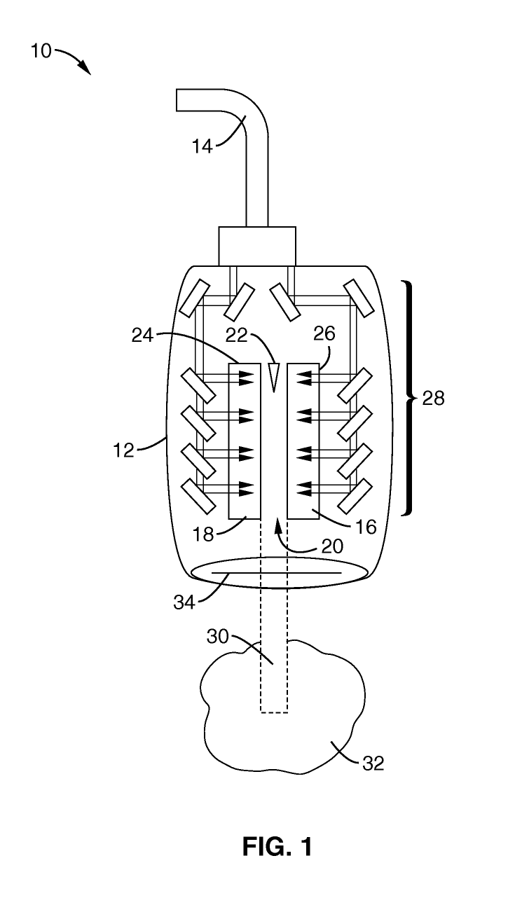

Referring more specifically to the drawings, for illustrative purposes the present invention is embodied in the apparatus generally shown in FIG. 1 through FIG. 7B. It will be appreciated that the apparatus may vary as to configuration and as to details of the parts, and that the method may vary as to the specific steps and sequence, without departing from the basic concepts as disclosed herein.

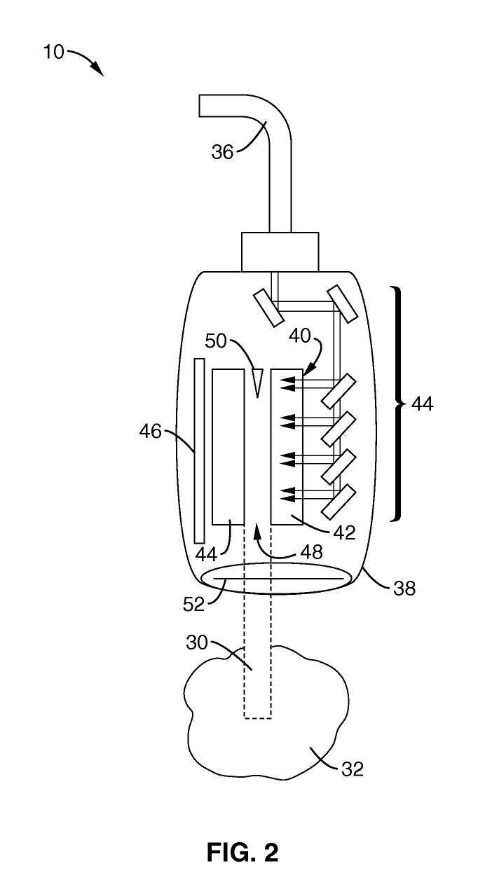

Turning now to FIG. 1 and FIG. 2, two embodiments of a Micro-Accelerator Platform (MAP) designed to generate beams of high intensity relativistic or near relativistic electrons and, optionally, bremsstrahlung x-rays are schematically shown. The apparatus 10 and system generally includes a MAP encapsulated in a housing 12 that is sized to attach to standard endoscope systems for use within the body of an animal or human patient. The entire structure is normally less than 1 cubic millimeter in size, and can be accommodated within a small disposable tip attached to a catheter. Although the invention is particularly useful for interior placement, it will be understood that the invention can be used externally wherever a beam of ionizing radiation can be beneficially used.

A source of laser light is provided that is preferably conducted to the housing 12 through a fiber optic cable 14 in the embodiments shown in FIG. 1 and FIG. 2. Laser light with a selected wavelength or range of wavelengths can be produced in the treatment room and transmitted through fiber-optic cable 14 down the catheter line to the accelerator. Thus electrons are produced, accelerated, and emitted entirely within the patient and can reach an energy range that is not currently available with existing brachytherapy sources. Therefore, the physician can place the radiation source right next to the tumor site and to deliver a controlled high intensity dose of ionizing radiation on to the tumor. After the required dose is delivered, the radiation can be turned off by shutting off the laser light source so that healthy tissues are not exposed during retrieval.

The accelerator of the embodiment shown in FIG. 1, has a pair of silicon wafers or slabs 16, 18, arranged side by side with a narrow vacuum gap 20 between the two wafers. An electron source 22 is located at the proximal end of the gap 20 and the distal end of the gap 20 is open. The wafers 16, 18 are preferably much wider than their separation distance 20, forming a ‘sandwich’ or ‘slab-symmetric’ geometry.

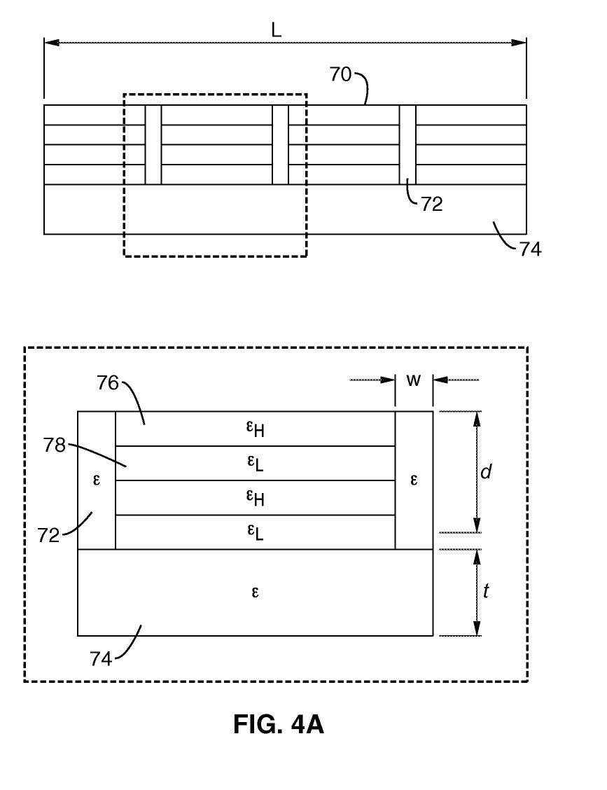

The outer surfaces 24, 26 of the two wafers 16, 18 are covered by at least one layer of reflective material and have a periodic array of slots filled with a dielectric as illustrated in FIG. 4A and FIG. 4B. Alternatively, the slots may be open to a vacuum. Laser light impinges on the structure from above and is directed into the vacuum gap 20 through the slots.

In the embodiment of the invention shown in FIG. 4A, alternating layers of differing dielectric are used in place of the reflective material, in a Bragg like structure configuration. The structure dimensions and other parameters are chosen to trap the laser radiation within the structure, causing a resonant buildup of the electric field in the gap 20 region.

The laser light from optical conduit 14 is distributed within the accelerator so that the laser light is directed to the outer surfaces 24, 26 of wafers 16, 18 and is illustrated conceptually in FIG. 1 and FIG. 2 as rays reflecting from a number of micro mirrors 28 to the wafer surfaces. Although the wafers 16, 18 could be illuminated with a series of milled reflective surfaces, it will be understood that laser light can be provided in many different ways such as directly orienting functional fiber optic surfaces with the surfaces 24, 26 of wafers 16, 18.

The vacuum gap 20 within the device 10 has an electron source 22 at one end that produces electron with initial velocity ˜0.3 times the speed of light that are ultimately accelerated through gap 20 and emitted as beam 30 to treat the tumor tissue 32. As the electrons gain energy from the laser field, the wafer structure and gap 20 is preferably tapered so that the phase velocity of the accelerating field increases to match the electron velocity. After traversing approximately 500 structural periods, the electrons are emitted from a proximal end 34 of the housing structure 12 with energies near 1-2 MeV, in one embodiment.

It will be seen that the geometry of wafer 16, 18 avoids many of the limitations of standard linear accelerators known in the art. High fields are confined to the vacuum/dielectric region 20 or kept away from the metal boundaries in the embodiment shown in FIG. 2 and transverse wakefields are suppressed. In addition, dielectric materials can survive very high electric fields without breakdown for short periods and dielectric construction allows for micro-machining and layering methods which can build small structures with extremely high precision.

An alternative embodiment of the MAP structure is show schematically in FIG. 2. In this embodiment, the dielectric wafers of the electron accelerator have a different structure than shown in FIG. 1. Referring now to FIG. 2, an optical conduit 36 is connected to the unit housing 38 and to a source of laser light (not shown) that can have the wavelength, intensity and other characteristics of the laser light controlled externally. The optical conduit 36 is preferably a flexible fiber optic cable sized for insertion with the housing 38 by catheter.

Laser light is transmitted through conduit 36 to the exterior surface 40 of a slab or wafer 42 through a series of reflecting surfaces 44 shown functionally in FIG. 2. One slab 42 is quasiperiodic having a reflecting surface (Bragg like stack) 40 interrupted every length p by a slot of width w, with p being a slowly varying function of axial position. The slot depth d is identical to the thickness of the reflecting surface. On the interior side of the reflecting surface is a uniform layer of dielectric material, having thickness t and dielectric constant ∈ as seen in FIG. 4A and FIG. 4B.

The other slab 44 is a dielectric disposed on a reflector 46 and does not have coupling slots as seen in slab 42. The slab structures 42, 44 may be parallel and separated by a vacuum gap 48 of width g.

An electron source 50 is located at one end of the vacuum gap 48. In one embodiment the electron source 50 comprises a ferroelectric crystal (FEC) overlaid with a deposited electron-emitting grid. A ferroelectric crystal such as lithium niobate will, when heated, spontaneously become charge polarized, giving rise to a normally-oriented surface electric field on the order of megavolts per centimeter. This pyroelectric effect produces a relatively long-lived field (relaxation time of several seconds).

Electrons can be emitted from the overlaid grid through field driven emission or in another embodiment through photoemission. The long-lived pyroelectric surface field will act as a constant-field acceleration region, causing the electrons to leave the cathode region with kinetic energies on the order of 28 keV.

In another embodiment, an end panel 52 is provided that is made of a material that will emit x-rays from the impact of accelerated electrons from gap 48. Such materials include tungsten, lead, gold and the like.

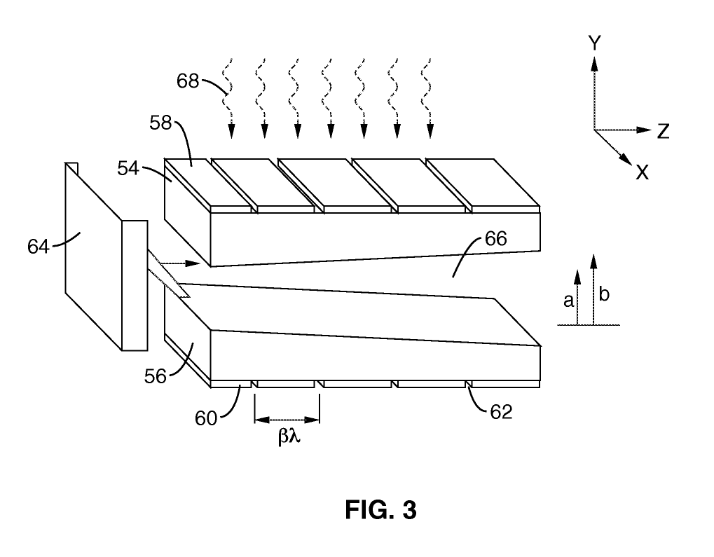

Turning now to FIG. 3, a schematic of the wafer/slab structures and cathode electron source of the accelerator are shown of the paired slotted dielectric configuration of the invention. In this embodiment, the accelerator has a pair of wafers, each having a dielectric base 54, 56 with at least one layer of a reflective surface 58, 60 interrupted by slots 62 in a preferably periodic array. Slots 62 can be filled with a dielectric or may be open to the vacuum of the housing. Laser light 68 is directed to the external surfaces of the paired wafer structures.

The symmetrical slab structures that are shown in FIG. 3 are separated by a gap 66. The gap 66 between the dielectric layers 54, 56 can be uniform and variable. However, a taper in gap 66 is preferred that is generally determined by consideration of the spacing between slots 62 and the width of the dielectric layers 54, 56.

The gap spacing g has no fixed value, but is linked to the dielectric thickness t and the material dielectric constant c (see below) of the base dielectric slabs 54, 56. Larger g values produce a larger electron beam aperture and makes beam injection and acceleration easier without boundary impacts. However, values of g that are greater than the approximate value of λ, result in high fields within the dielectric layers and significant field nonuniformity for β<0.5. Therefore, a value of g=λ is the most effective compromise, producing tolerable fields with a realistic electron aperture. Adjusting the gap is also the easiest way, in practice, to tune the structure.

An electron source 64 is provided that is capable of injecting electrons for acceleration within the gap 66 between the two slabs. An integrated particle emitter gun is used in one embodiment of the accelerator 10. The function of the gun 64 is to produce a stream of electrons of sufficient intensity and energy to be trapped and accelerated by the fields in the remainder of the structure. There are two stages of operation: (1) electron emission and (2) acceleration up to the threshold β₀.

Ideally, the emission time of the gun 64 would be well matched to the structure cycle time (on the order of the fill time and laser pulse length). However, in practice, electrons emitted at the wrong time (phase) will be untrapped and unaccelerated, or will quickly become untrapped. In principle, the gun can operate by field emission, photoemission (i.e. photoelectric effect), or thermionic emission.

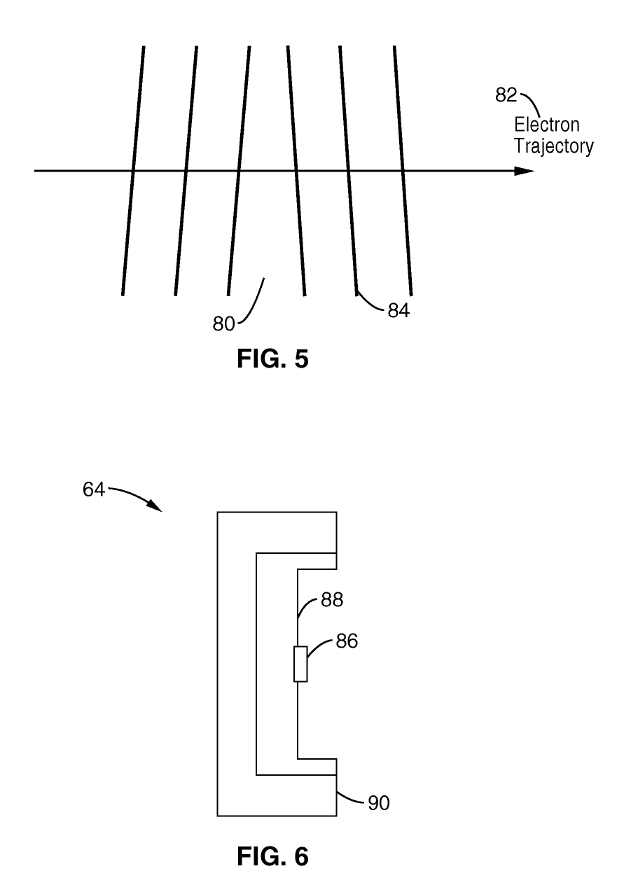

Acceleration of the electrons from the cathode 64 surface to the necessary β₀ (e.g. 25 keV) can be accomplished through an externally applied field, as is typically done in a DC gun. However, in practice, it is preferable to eliminate the external high voltage source. Such a gun is producible using the internal fields found in pyro-electric crystals. In such crystals, it is possible to produce electric fields of tens of KV at the surface of crystals such as LiNbO₃ and LiTaO₃. The gun then consists of a modest heater used to cycle the temperature of the field production crystal, and a second crystal or field emitter which produces the electrons as shown in greater detail in FIG. 6.

Details of one embodiment of the slab or wafer structure 54,56 are schematically shown in FIG. 4A and FIG. 4B to illustrate the general structure of one type of slab. The structure shown in FIG. 4 is intended to show one possible multiple layer embodiment and is not drawn to scale. The accelerator preferably has micro scale dimensions of approximately 1 mm or less per side so that it can be delivered to locations of the body by a catheter yet the stream of ionizing radiation will be of therapeutic intensity and localized in origin and dispersion. The dimensions of the slab structure can be selected to produce an electron beam of desired characteristics as well as account for manufacturing efficiencies and material limitations.

The overall slab dimensions (length L and width W) are not critical parameters and need only to be big enough to prevent edge effects from disturbing the acceleration. Poor choices will degrade the performance of the accelerator but will not prevent operation.

Overall length L: Should optimally be np, where n is the number of periods, typically on the order of 1000. The number of periods n is normally set by the field gradient and desired output energy and p is the spacing between slots.

Overall width W: Should be much larger than any other dimension affecting the field, as well as larger than the electron emitting region (i.e. the electron gun). If W>>g, this condition is satisfied. Hence W can range from approximately ten to approximately 1000 micrometers.

A side view of a slab structure is provided in FIG. 4A of an embodiment where the slots are filled with a dielectric material. This dielectric material can be composed of the same material as the base slab dielectric. Alternatively, the slot dielectric can be a dielectric that is different from the slab dielectric. Furthermore, the slot dielectric structure can be eliminated and open to the vacuum of the enclosure in one embodiment.

Each slab has a base dielectric 74 having a thickness t. Many different types of dielectric materials may be used for the slabs in this device. Material selection involves consideration of the transmission at operating wavelength of the material and the complex index of refraction (dielectric constant) at wavelength which includes the so-called loss tangent; breakdown voltage as well as the deposition and crystallization properties of thin films of the material. The ideal material has, at the operating wavelength, high transmission (>0.9), a high index of refraction (>1.5), low loss tangent, high breakdown voltage (>100 MV), and can be formed into epitaxial or single crystal films.

The preferred fabrication processes and the selection of suitable materials are similar to those used in microchip and microstructure (MEMS) fabrication. Silicon possesses many favorable qualities for the slabs, but is opaque at wavelengths shorter than about 1.2 μm. Silicon carbide (SiC) has transmission in the desirable 800-1064 nm band; superior breakdown voltages; and superior thermal properties (for handling high average powers); but is generally inferior to silicon in terms of ease of fabrication and availability of quality bulk materials. Glasses such as fused silica, quartz and sapphire provide excellent bulk and surface qualities and possess acceptable breakdown voltages, but have lower indices of refraction and therefore may produce lower efficiency structures. Finally, diamond is perhaps the ideal slab material but is expensive and difficult to mass produce.

Dielectric constant ∈: The dielectric constant of the base 74 is determined by the material that is selected and used to line the interior of the slabs forming the gap. Higher values of ∈ lead to more efficient structures since less field is confined within the dielectric in these structures. In practice, however, the range of ∈ values for possible micromanufacturing materials is not large, with most materials having values from 2 to 4; silicon carbide (∈=6.8) is among the highest practical choices.

Dielectric thickness t: The thickness t of the base dielectric 74 is preferably fixed once gap spacing g and dielectric constant ∈ of the material are determined, via the formula:

$$\frac{γ β}{ɛ} \sqrt{ɛ - \frac{1}{{β}^{2}}} = cot ⌊ \frac{ω t}{c} \sqrt{ɛ - \frac{1}{{β}^{2}}} ⌋ coth [ \frac{ω g}{γ β c} ]$$

where ω is the laser angular frequency and γ is the electron relativistic factor (1−β²)^{1/2}. This formula, however, is exactly correct only for an infinitely wide structure with no coupling slot perturbations, so t may need to be corrected via simulation for structures with physical coupling. Furthermore, t will also vary as β increases.

Slot width w: The width of the slots 72 shown in FIG. 4A and FIG. 4B does not have to be calculated analytically. However, it is normally necessary to have w<<p for reasonable field confinement within the structure. Slots 62 both allow laser power 68 to couple into the structure and perturb the accelerating electron field in their vicinity (as well as the resonant frequency) within the gap 66 between the slab structures as illustrated in FIG. 3.

Wider slots 72 give better coupling (more efficient use of laser energy) but more perturbation (less mode purity). Therefore, the selection of the optimal slot 72 width is a compromise. Slot dimensions may also be constrained by the limits of easy manufacturability. Simulations showed a broad optimum around λ/10. For an 800 nm design, for example, a compromise value of about 50 nm may be chosen, but values of w from 10 nm to 100 nm were also shown to be functional.

Slot depth d: The theoretical optimal value of the depth d of the slot 72 is one that makes an ideal impedance match. For example, for any waveguide coupler, a length of exactly one-quarter wavelength will not perturb the cavity fields. In this context, the ideal slot depth d can be evaluated with the following:

$${d}_{ideal} = {λ}_{g} ( \frac{1}{4} + m ) , \qquad m = 0 , 1 , 2 , 3 …$$

where λ_{g} is the appropriate free-space laser wavelength. (The slot may be filled with vacuum or with a dielectric, which in the latter case would reduce the field amplitude within the slot. In either case, the value λ_{g} is the laser wavelength in the material.) If d is ideal, there is no perturbation of structure fields. However, manufacturing concerns like large aspect ratios make the use of ideal slots unattractive in some applications. Simulations have shown that far smaller values of d than the calculated ideal work well if the vacuum gap g is adjusted slightly to compensate for the small detuning that arises. For the 800-nm design, for example, a slot depth of 80 nm produces acceptable field nonuniformity in simulation (less than 5%) while reducing the slot aspect ratio from 1:4 (i.e. 50 nm: 200 nm) to less than 1:2, greatly easing fabrication constraints.

The base dielectric 74 shown in FIG. 4A, has a number of alternating layers of high 76 and low 78 index of refraction materials that is bounded by slots 72 and collectively having a thickness that is equal to the slot depth d. The alternating layers 76, 78 preferably range in thickness from approximately 50 nm to approximately 300 nm. The number of layers can vary and is determined primarily by the quality of fabrication to provide the desired characteristics. Typically, nine or more layers of high 76 and low 78 index refraction materials are used and disposed on 74 the base dielectric of the slab. The quality of the Bragg structure primarily affects the efficiency of the device, which is not a central concern. Furthermore, additional laser power can be used up to the point where structure heating is too great.

Fabrication methods for producing alternating thin films or layers of material on a substrate are well developed in the art. For example, Bragg type reflector stacks (needed for this all-dielectric device) have been commercially produced from a wide range of high- and low-index of refraction materials. One common “sandwich” used in nano-lasers is the InGaAsP stack. In bulk optics, films of oxides and florides are commonly used (e.g. MgF₂). Techniques developed for producing vertical cavity surface emitting lasers (VCSEL) and other photonic bandgap (PBG) structures utilizing machined layers of thin laminates of materials can also be used to manufacture the slab structures of the present invention.

As seen in the top view of FIG. 4B, the spacing p of reflecting surface 70 between slots 72 is preferably fixed at the value of the free-space laser wavelength λ multiplied by the normalized electron velocity β; i.e. p=βλ. The choice of a particular laser and the anticipated electron velocity in the structure hence determine the value of p, which will increase quickly in the beginning and slowly towards the end of the structure as the electrons accelerate.

Therefore, essentially any laser frequency can be selected and that selection will usually be guided primarily by commercial availability of the laser. Common choices are lasers with wavelength λ=800 nm, 1064 nm, 1550 nm, and 10 μm. The laser that is selected should be able to sustain the necessary pulse repetition rate, and the optical properties (e.g. losses) of the slab material and substrate must be reasonable at that frequency.

The value of β at any point in the slab structure can be found given the injection energy of electrons and field strength on axis. The field strength on axis generally sets the (roughly constant) energy gain per unit length of the electrons. Mathematically, the ideal resonant trajectory has

$${β}_{r} = \sqrt{1 - \frac{1}{{( 1 + {β}_{0} + Az )}^{2}}}$$

where β₀ is the injection velocity and A is the acceleration per unit length, in suitable units. For stable injection, β₀²≧1−1/∈. The number of periods is thus determined by the desired output energy and the value of A. The gradient A is proportional to the field strength of the incoming laser, which is limited primarily by the electric breakdown threshold of the reflectors and dielectric substrate. This limit is well characterized in general, but is ideally determined experimentally for short pulses (less than 1 pico second) in this geometry, but is believed to be at least 1 GV/m.

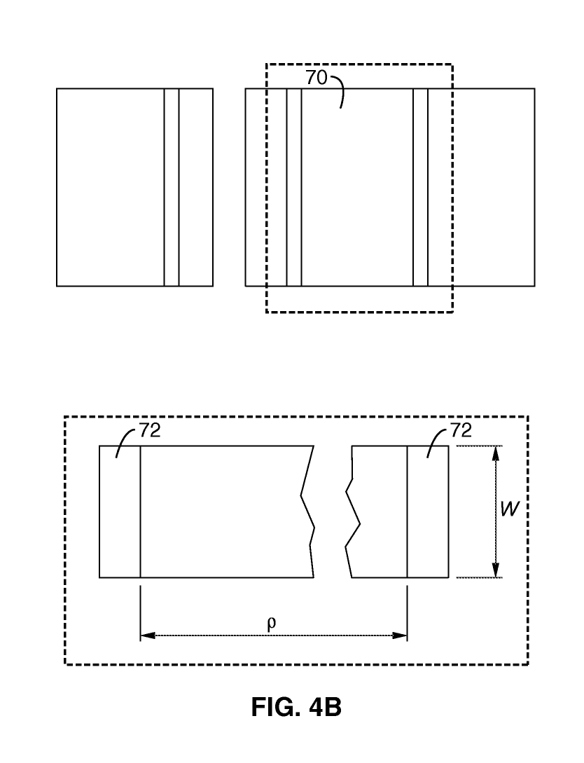

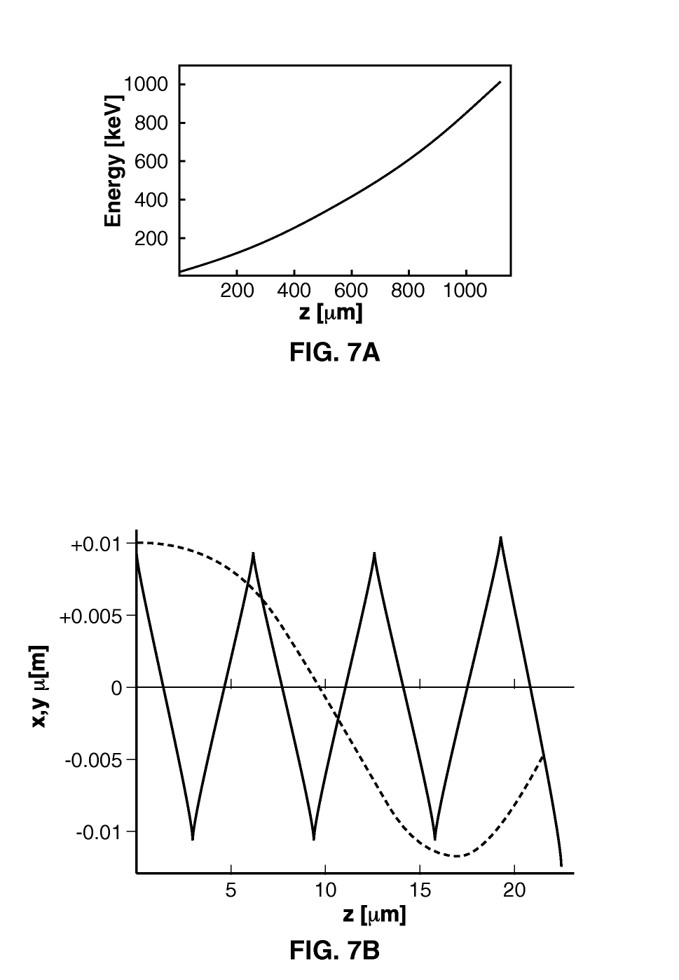

FIG. 5 is an illustration of an alternative embodiment of the slab structure 54, 56 shown in FIG. 3 viewed from the top. The slab structure seen in FIG. 5 has an upper surface 80 with periodic slots 84. The trajectory 82 of the electrons accelerated along the lower dielectric layer is shown for reference. In this embodiment, stable acceleration of electrons over hundreds of periods is accomplished using a canted structure which maintains focusing in the small (y) direction while alternating transverse kicks in the (x) direction as oriented in FIG. 3. The coupling slots 84 are rotated by a small, preferably β-dependent angle from perpendicular, in effect using a nonzero transverse velocity to oppose the defocusing kick Fₓ. The slots 84 are alternated along the length of the slab structure. After several structure periods, when the electron has crossed the center line, the slot angles change to the opposite sign as illustrated in FIG. 7B.

One embodiment of an integrated electron source 64 of FIG. 3 is shown in FIG. 6. Electrons are generated by field emission and then accelerated in a quasi-DC electric field to approximately 25 keV or greater. The cathode design shown conceptually in FIG. 6 has a small field emitting region 86, such as an array of graphite needles, deposited on a ferroelectric crystal base 88, such as lithium niobate (LiNbO₃) or LiTaO₃. Ferroelectric crystals (FEC) typically have pyroelectric properties so that they develop a temporary polarization on the crystal surface when heated or cooled. The temporary polarization charge that is developed has been shown to be proportional to the temperature increase and the pyroelectric coefficient of the material. The polarization charge is eventually neutralized by bulk conduction in the material. However, that process normally has a neutralization time that is several seconds in duration.

The total energy gained from an electron emitted from 86 and accelerated in the surface field depends on the size and characteristic of the ferroelectric crystal 88. For example, a circular lithium niobate FEC preferably has a radius of approximately 0.5 mm.

Accordingly, in the embodiment shown on FIG. 6, the cathode operation is a two stage process. First, the cathode is heated by a heater 90 to provide a quasi static DC field. Then the cathode yields electrons through field emission from the tips of the emitter 86. The electrons are injected into the gap 66 between the slabs and accelerated. Normally, a gap of less than a millimeter between the cathode 64 and the acceleration structure will permit the injection of electrons at high enough energy into the gap for trapping and acceleration.

The invention may be better understood with reference to the accompanying example, which are intended for purposes of illustration only and should not be construed as in any sense limiting the scope of the present invention as defined in the claims appended hereto.

### Example 1

To demonstrate the function of the microscale particle accelerator, a resonant laser powered structure measuring 1 mm or less in every dimension that is capable of generating and accelerating electron beams at 1-2 MeV energies was evaluated. The accelerator structure had a pair of parallel dielectric slabs separated by a narrow vacuum gap and bounded above and below by a reflective layer or layers. The slabs had a total length of 1 mm and had approximately 1600 structure periods. Periodic slots in the reflector were used to provide a means for coupling radiation into the gap and also to enforce longitudinal periodicity in the structure fields. The dimensions (vacuum gap and dielectric thickness) of the structure were selected so that the structure would be resonant at the laser frequency so that the field pattern would be dominated by a longitudinal standing wave with phase velocity (c). The accelerating field was shown to be typically 4 to 10 times larger than the incident laser field.

Since the structure dimensions will vary with beam velocity, the gap was tapered as the beam energy increased. The gap ranged from a=0.05 μm through 0.1 μm at the top of the taper and b=0.27 μm through 0.31 μm at the top as illustrated in FIG. 3. The structure was also modulated in the z direction by coupling slots that had a periodicity of 2π/k_{z} and the slot spacing was tapered and equal to βλ, where λ was the free-space laser wavelength.

One approach permitting stable acceleration over hundreds of periods using a canted coupling slot structure that maintained focusing in the (y) direction while alternating transverse kicks in the (x) direction was evaluated.

The structure was evaluated using single particle tracking through analytic fields. As seen in FIG. 7A, particle energy along the structure is shown assuming a 3.5 GV/m field strength within the gap from a GW class laser. It can be seen that the energy gain for a particle on the axis appears to be smooth with an output energy of 1 MeV reached in just over 1 mm of travel.

Focusing the canted-slot structure showing values of x and y in the first 20 periods of the structure is shown in FIG. 7B. The structure is focusing in the y direction (dashed line) and alternates defocusing kicks in x (solid line).

Accordingly, a micro scale relativistic slab-symmetric dielectric based electron accelerator is provided that is capable of generating electron beams or x-rays. The scale of the apparatus permits adaptation to catheter systems for placement in otherwise inaccessible the body, for example, and the simple design allows construction from conventional microfabrication techniques

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A micro-accelerator platform, comprising
an electron source;
a first dielectric slab with a reflective surface having a plurality of slots and an active surface;
a second dielectric slab with a reflective surface having a plurality of slots and an active surface, said active surface oriented opposite said active surface of said first dielectric slab forming a gap between said active surfaces; and
a source of optical radiation configured to direct beams of light on said reflective surfaces of said first and second dielectric slabs;
wherein electrons emitted from the electron source are accelerated within said gap between the active surfaces of the first and second dielectric slabs.

2. A platform as recited in claim 1, wherein said slotted reflective surface of said first and second dielectric slabs comprises a plurality of alternating layers of high index and low refractive index dielectric material.

3. A platform as recited in claim 1, wherein said slotted reflective surface of said first and second dielectric slabs comprises a metallic reflector.

4. A platform as recited in claim 1, further comprising:
means for focusing a stream of accelerated electrons.

5. A platform as recited in claim 3, wherein said means for focusing a stream of accelerated electrons comprises alternating series of canted slots in the reflective surface of said first and second dielectric slabs.

6. A platform as recited in claim 1, wherein the active surfaces of said first and second dielectric slabs are oriented so that the gap between the slabs is tapered.

7. A platform as recited in claim 1, further comprising:
means for converting a stream of electrons to x-rays.

8. A micro-accelerator platform, comprising
an electron source;
a first dielectric slab with a reflective surface having a plurality of slots and an active surface;
a second dielectric slab with a reflective surface and an active surface, said active surface oriented opposite said active surface of said first dielectric slab forming a gap between said active surfaces; and
a source of optical radiation configured to direct beams of light on said reflective surface of said first dielectric slab;
wherein electrons emitted from the electron source are accelerated within said gap between the active surfaces of the first and second dielectric slabs.

9. A platform as recited in claim 8, wherein said slotted reflective surface of said first dielectric slab comprises a plurality of alternating layers of high index and low refractive index dielectric material.

10. A platform as recited in claim 8, wherein said slotted reflective surface of said first and second dielectric slabs comprises a metallic reflector.

11. A platform as recited in claim 8, further comprising:
means for focusing a stream of accelerated electrons.

12. A platform as recited in claim 11, wherein said means for focusing a stream of accelerated electrons comprises alternating series of canted slots in the reflective surface of said first dielectric slab.

13. A platform as recited in claim 8, wherein the active surfaces of said first and second dielectric slabs are oriented so that the gap between the slabs is tapered.

14. A platform as recited in claim 8, further comprising:
means for converting a stream of electrons to x-rays.

15. A radiation source, comprising:
an evacuated housing; and
a micro-accelerator platform assembly disposed within said evacuated housing, said platform assembly comprising:
a pair of dielectric slabs separated by a vacuum gap, each slab having a reflective layer on a side opposite said gap, with at least one reflective layer having a plurality of periodic slots;
a source of electrons configured to emit electrons within said vacuum gap; and
an optical source adapted to directing beams of light to said reflective layers of said dielectric slabs;
wherein electrons from said electron source are accelerated.

16. A radiation source as recited in claim 15, further comprising:
a vascular access system adapted to deliver the micro-accelerator platform assembly into the body to a location within the body.

17. A radiation source as recited in claim 16, wherein said vascular access system includes a flexible fiber optic catheter.

18. A radiation source as recited in claim 15, wherein said slotted reflective surface of said first and second dielectric slab comprises a plurality of alternating layers of high index and low refractive index dielectric material.

19. A radiation source as recited in claim 15, wherein said beams of light are directed perpendicularly to said slotted reflective surfaces of said dielectric slabs by mirrors.

20. A radiation source as recited in claim 15, wherein said beams of light are directed perpendicularly to said slotted reflective surfaces of said dielectric slabs by fiber optic cables.

21. A radiation source as recited in claim 15, further comprising:
means for converting a stream of electrons to x-rays.

22. A radiation source as recited in claim 21, wherein said means for converting a stream of electrons comprises a lead plate.

23. A radiation source as recited in claim 15, said electron source further comprising:
a ferroelectric crystal base;
an emitter array coupled to said ferroelectric crystal base; and
a heating element.

24. A radiation source as recited in claim 23, wherein said emitter array comprises graphite needles.

25. A radiation source as recited in claim 23, wherein said ferroelectric crystal base comprises lithium niobate.
